(19) United States
(12) Patent Application Publication
(10) Pub. No.: US 2010/0024247 A1
(43) Pub. Date: Feb. 4, 2010

(54) PROTECTIVE APPLIANCE FOR FLIP-FLOP TYPE FOOTWEAR

(76) Inventors: Daniel T. Feeney, Merrimac, MA (US); John C. Hughes, Canton, MA (US); Victor M. Souza, Billerica, MA (US); Timothy G. Hickey, South Easton, MA (US)

Correspondence Address: CESARI AND MCKENNA, LLP, 88 BLACK FALCON AVENUE, BOSTON, MA, 02210, US

(21) Appl. No.: 12/185,497

(22) Filed: Aug. 4, 2008

(51) Int. Cl. A43B 7/26; A43B 7/14; A43B 23/00; A43B 3/12; A61F 13/06

(52) U.S. Cl. 36/94; 36/88; 36/136; 36/11.5; 128/894

(57) ABSTRACT

A protective appliance for the space between the toes of a wearer of flip-flop sandals or other such footwear. The appliance has a generally hollow, generally elongated cylindrical shape and is fabricated from a relatively light weight, soft and compressible material. It includes a longitudinal cut through one of its sides that allows the appliance to be opened and engaged about the thong segment of the flip-flop that normally extends between the large toe and adjacent toe of the wearer, and that resiliently closes after the appliance is so engaged. When applied, the appliance cushions the space between the toes and the thong segment and allows at least a limited degree of both longitudinal and radial movement of the appliance relative to the thong segment thus increasing comfort of the flip-flops and minimizing friction and chafing of the toes on the thong segment during walking and other activities in the flip-flops. The appliance may be readily removed from the thong segment and moved from one pair of flip-flops to another as desired.

### BACKGROUND OF THE INVENTION

1. Field of the Invention

This invention relates to a protective appliance for the feet, and more particularly, to an appliance for protecting the toes of a wearer of flip-flop sandals and other such footwear from irritation and chafing during walking and other activities in the flip-flops.

2. Background Information

Flip-flops have become increasing popular in today's culture as the footwear of choice for leisure, casual and recreational occasions. The conventional flip-flop includes a base sole portion generally conforming to the shape of the bottom of the foot, and a thong or strap portion attached to and extending upwardly from the sole portion. The thong portion of the flip-flop typically includes a thong segment adapted to engage between the large toe and adjacent toe of the wearer when worn and side segments extending outwardly in opposed directions from the upper end of the thong segment and adapted to extend along the top and sides of the foot then reattach to the sole portion just forward of the heel area thereof. The placement of the thong segment between the toes, and engagement of the side segments along the top and side of the foot, retain the foot in the flip-flop during walking and other activities.

By their very design, flip-flops are generally only loosely retained on the wearer's foot. During walking or other activities, the wearer's foot moves to some degree relative to the flip-flop structure. The impact of this movement is typically most problematic in the space between the large and adjacent toes occupied by the thong segment extending up from the flip-flop sole portion. That area of the foot is relatively sensitive compared to other areas of the foot and is thus particularly susceptible to irritation and chafing due to the abrading action of the moving thong segment on the skin between the toes. For this reason, many individuals find flip-flops with between-the-toe thong segments to be too uncomfortable to wear. Others wear the flip-flops only during lounging and shun them during times and activities that are generally more active or athletic. It would be desirable to provide an appliance that could be used in conjunction with conventional flip-flops to minimize their inherent discomfort and expand the times when, and circumstances under which, they can be conveniently and comfortably worn.

One prior art approach to this problem is disclosed in U.S. Pat. No. 7,335,178 (the “'178 patent”). The '178 patent discloses a device which is adapted to fit in the space between the large toe and adjacent toe of the foot to provide protection from frictional injury to that area when wearing thong-type sandals. The device has a flat base portion that fits under the toes and on the bottom of the foot, a web portion that extends upwardly from the base portion and fits between the toes, shielding them from frictional contact with the thong strap of the sandal, and a flat upper portion that extends rearwardly from the web portion and anchors the device to the top of the foot.

The device of the '178 patent has a relatively complex shape and configuration, making it relatively expensive to manufacture. Additionally, by its very design, the '178 patent device is intended to, in effect, clip onto the user's foot. Thus, when the user removes his or her foot from the sandal, the device remains anchored to the foot. This would be considered undesirable to many users, not only from an appearance standpoint, but also due to the inconvenience it creates in regard to either maintaining the device in position on the foot or having to remove then reinsert it, when the sandals are removed.

It is, therefore, an object of the present invention to provide an improved protective appliance for a flip-flop sandal or other such footwear which makes the sandal generally more comfortable to wear and which protects the wearer's toes from injury due to frictional contact with the thong strap engaged between the toes.

It is another object of the present invention to provide an improved protective appliance of the type described that may be removably attached to the thong strap segment of each flip-flop in one pair, and then removed and removably attached to the thong strap segment of each flip-flop in another pair, thus allowing the appliances to be used interchangeably with multiple flip-flop pairs.

It is another object of the invention to provide an improved protective appliance of the type described that conveniently remains attached to the thong strap segment of the flip-flop when the wearer removes his or her foot from the flip-flop.

It is still another object of the invention to provide an improved protective appliance of the type described that is unobtrusive and attractive in appearance.

It is still yet another object of the invention to provide an improved protective appliance of the type described that is relatively simple in design and relatively inexpensive to manufacture.

### SUMMARY OF THE INVENTION

The present invention provides an improved protective appliance for the space between the toes of a wearer of flip-flop sandals or other such footwear which meets each of the above-described objects of the invention.

In accordance with a first embodiment of the invention, a protective appliance is provided that has a generally hollow, generally elongated, generally cylindrically-shaped body fabricated from a relatively light weight, soft and compressible material. The appliance body may include a thin, inner layer that is relatively flexible and relatively resilient, and a thicker outer layer that is relatively soft and relatively compressible. A cut is provided longitudinally through a side wall of the appliance body (e.g., through both the inner and outer layers thereof) to allow the appliance to be opened so that the thong-strap segment of the flip-flop that normally extends upwardly between the large toe and adjacent toe when the flip-flops are worn may be engaged in the inner hollow space of the body. The resiliency of the inner layer returns the appliance to its closed position and holds the appliance securely on the thong-strap segment until such time as the appliance is to be removed. The soft, compressible outer layer provides a cushioned interface between the thong strap segment and the skin between the toes of the wearer. The hollow space inside the appliance body is preferably sized relative to the thong-strap segment so that the appliance, when installed, can move at least to a limited degree both radially and longitudinally relative to the segment. This freedom of movement of the appliance minimizes the possibility of abrading and chafing of the skin between the toes on the thong-strap segment during walking and other activities in the flip-flops.

The appliance can readily be removed from the flip-flop when desired simply by pulling the appliance away from the thong-strap segment until the segment passes through the opening in the side wall thereof. The appliance can then be reinstalled on another pair of flip-flops or stored for later use.

Various alternative embodiments of the appliance of the invention are disclosed.

### BRIEF DESCRIPTION OF THE DRAWINGS

The foregoing and other objects, features and advantages of the invention will be better understood from the following detailed description of illustrative embodiments of the invention, taken in conjunction with the accompanying drawings, in which:

FIG. 1(a) is a top plan view of a conventional flip-flop style sandal;

FIG. 1(b) is a side plan view of the conventional flip-flop sandal of FIG. 1;

FIG. 2(a) is a top plan view of a protective appliance embodied in accordance with the invention;

FIG. 2(b) is a side cross-sectional view of the protective appliance of FIG. 2(a);

FIG. 3 is a top view showing the protective appliance of FIGS. 2(a) and (b) in a partially open state about to be engaged about a thong-strap segment of a flip-flop sandal;

FIG. 4 is a side plan view of the flip-flop sandal of FIGS. 1(a) and 1(b) with the protective appliance of FIGS. 2(a) and (b) applied thereto;

FIG. 5(a) is a top plan view of a second embodiment of the protective appliance of the invention;

FIG. 5(b) is a side plan view of the second embodiment of the protective appliance shown in FIG. 5(a); and

FIG. 6 is a top plan view of a third embodiment of the protective appliance of the invention.

### DETAILED DESCRIPTION OF ILLUSTRATIVE EMBODIMENTS

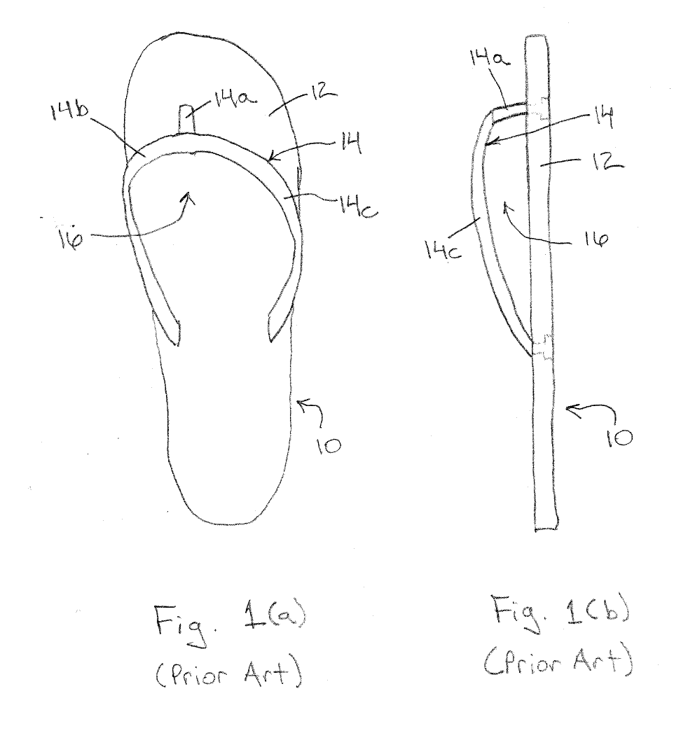

FIGS. 1(a) and (b) illustrate a conventional flip-flop sandal 10 of the type with which the protective appliance of the present invention is used. The flip-flop 10 includes a sole or base portion 12 having a shape generally conforming to the bottom of the foot of a human wearer. A thong or strap portion 14 is attached at three spaced points to the upper side of the sole portion 12 by conventional means and provides an opening 16 above the sole portion 14 to accommodate the wearer's foot. Specifically, the strap portion 14 includes a toe segment 14(a) attached to a forward part of the sole portion 12 and adapted to fit between the large toe and adjacent toe of the wearer's foot. The strap portion 14 also includes a pair of counterextending side strap segments 14(b) and (c) that are integral with the upper end of the toe segment 14(a) and that extend in opposed directions initially to the opposite sides of the sole portion 12 and then toward the rear of the sole portion 12 where they attach to opposite sides of the sole portion 12 just forward of the heel area of the same. The engagement of the toe segment 14(a) between the wearer's toes, and positioning of the side strap segments 14(b) and 14(c) along the top and sides of the foot, loosely retain the flip-flop 10 on the wearer's foot.

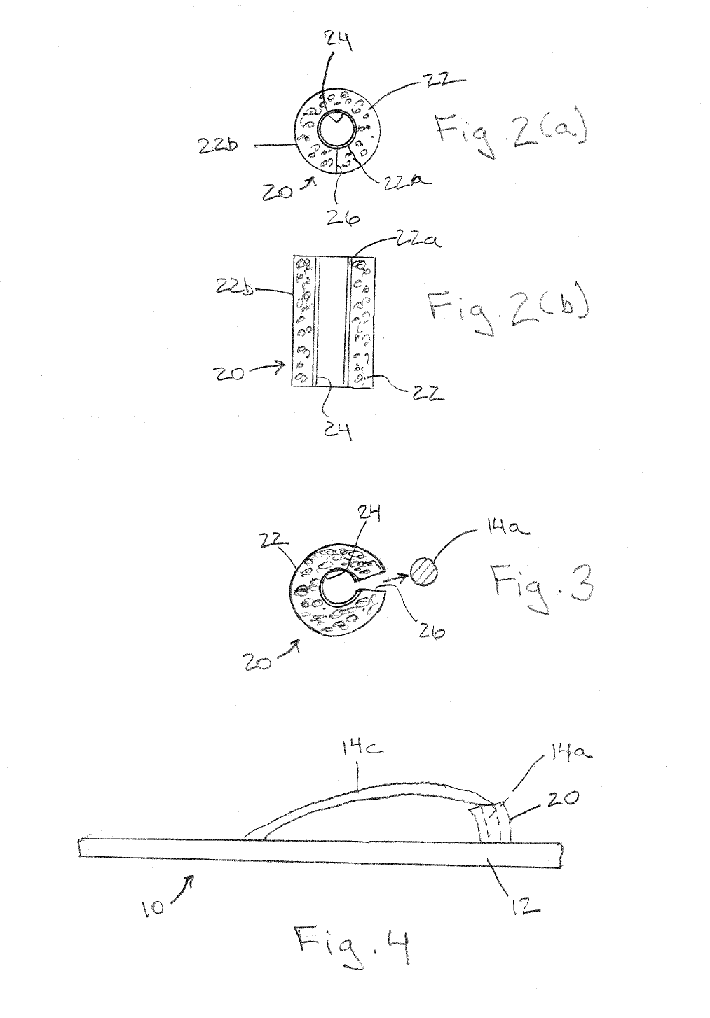

FIGS. 2(a) and 2(b) illustrate a first embodiment of a protective appliance 20 in accordance with the present invention. Illustratively, the protective appliance 20 includes a generally hollow, generally elongated, generally cylindrically-shaped body 22 that defines an inner or central space 24. The body is comprised of a thin, inner layer 22(a) of relatively flexible, relatively resilient material, such as polyethylene plastic, and a thicker, outer layer 22(b) of a relatively soft, relatively compressible material, such an open-cell polystyrene foam. The inner layer 22(a) may be secured to the outer layer 22(b) by any conventional means such as through the use of heat sealing, an adhesive or the like. Alternatively, the inner layer 22(a) may be formed by treating the inner surface of the outer layer 22(b) chemically or by other known means to increase its hardness and resiliency.

The body 22 of the protective appliance 20 is provided with a cut 26 that runs longitudinally along its side wall generally parallel to the central or inner space 24 in the body 22. The cut 26 allows the body 22, including its outer layer 22(b) and inner layer 22(a), to be pried open to allow access to the space 24. This allows the protective appliance 20 to be installed about the toe segment 14(a) of the flip-flop 10, as illustrated in FIG. 3 of the drawing. The space 24 in the body 22 of the protective appliance 20 is preferably sized so that, when installed, the protective appliance 20 is free to move at least to a limited degree both radially and longitudinally relative to the toe segment 14(a). The resiliency of the inner layer 22(a) of the body 22 is such that, when the appliance 20 is installed on the toe segment 14(a) of the flip-flop 10, it closes the cut 26, retaining the appliance 20 on the toe segment 14(a) until it is to be removed.

FIG. 4 illustrates the protective appliance 20 attached to the toe segment 14(a) of the flip-flop 10 of FIGS. 1(a) and (b). As indicated in FIG. 4, the toe segment 14(a) may have a curved or contoured shape sloping slightly to the rear or heel portion of the flip-flop 10. The appliance 20 may be flexible enough to accommodate this contour, or itself be contoured in shape, to insure a relatively loose fit of the appliance 20 on the toe segment 14(a).

The outer layer 22(b) of the protective appliance 20 provides a cushioned interface between the wearer's toes and the toe segment 14(a) of the flip-flop 10, making the flip-flop 10 generally more comfortable to wear. The freedom of movement of the appliance 20 both radially and longitudinally relative to the toe segment 14(a) minimizes frictional injury to the wearer's toes due to movement of the toe segment 14(a) relative to the foot during walking and other activities in the flip-flop 10. The appliance 20 is also such that it remains attached to the flip-flop 10 when the wearer removes his or her foot from the flip-flop, avoiding the inconvenience associated with prior art devices that attach directly to the wearer's foot. Yet, when desired, the appliance 20 can be readily removed from the flip-flop 10, by pulling the appliance 20 away from the toe segment 14(a) until the toe segment 14(a) passes through the cut 26 in the appliance body 22. The appliance 20 can then be conveniently attached to another flip-flop of choice.

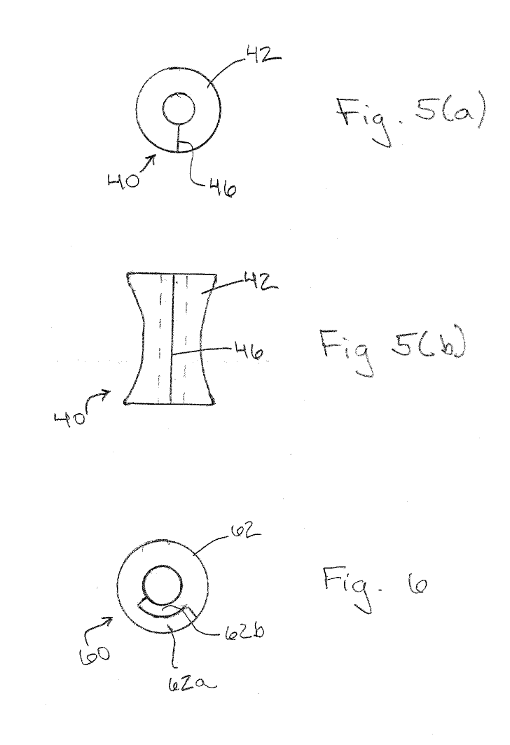

FIGS. 5(a) and (b) illustrate a second embodiment of a protective appliance 40 in accordance with the invention. The protective appliance 40, like the appliance 20 previously described, has a generally hollow, generally elongated body 42 with an inner or central space 44 and a longitudinal cut 46 through its side wall to allow the body 42 to be opened for installation about the toe segment 14(a) of a flip-flop 10. The body 42 is, however, of a solid, unitary construction molded or otherwise fabricated from a relatively compressible, relatively resilient material, such as a thermoplastic elastomer (TPE) material or a soft polymer gel. Materials such as these give the body 42 the desired compressibility for cushioning the wearer's toes and the desired resiliency to retain the appliance 40 on the toe segment 14(a) of the flip-flop 10 when the flip-flop 10 is worn, and when the wearer's foot is removed from the flip-flop 10. As illustrated in FIG. 5(b), the body 42 also has side walls that are concave, or recessed progressively near its central region, giving the body 42 a generally hour-glass-like shape. This shape also helps retain the appliance 40 in proper position on the toe segment 14(a) regardless of the movement of the foot relative to the flip-flop 10 during walking and other activities.

FIG. 6 illustrates a third embodiment of a protective appliance 60 in accordance with the invention. The appliance 60 includes a body 62 that is substantially identical to the body 22 of the appliance 20 or the body 42 of the appliance 40 previously described, except that the longitudinal cut through the side wall is provided by a pair of overlapping segments 62(a) and 62(b) of the side wall of the body 62. The overlapping side wall segments 62(a) and 62(b) can be pried opened to install the appliance 60 on the toe segment 14(a) of the flip-flop 10. The segments 62(a) and (b) remain closed after the appliance 60 is installed due to the resiliency of the material of which the body 62 is made. Alternatively, the contacting surfaces of the overlapping segments 62(a) and (b) may be provided with complementary hook and loop fastening strips, such as those offered commercially under the trademark Velcro®, to secure the segments together when the appliance 60 is installed on the flip-flop 10. The fastening strips can be separated from one another and the overlapping segments 62(a) and (b) pulled open when the appliance 60 is to be removed from the flip-flop 10.

Other modifications or additions to the above-described embodiments of the invention will now suggest themselves to those skilled in the art. For example, although the appliance bodies 22, 42 and 62 have been shown to have a height that is somewhat greater than their width or diameter, other body shapes can, of course, be provided including one in which the length of the appliance body between the wearer's toes is greater than its height and width. The appliance bodies 22, 42 and 62 may also be made available in a variety of colors and/or provided with decorative outer coverings or treatments to give the appliance an attractive or novel appearance that comports with that of the flip-flops with which they are used. Alternatively, the appliance bodies 22, 42 and 62 may be fabricated from a transparent material so that they are barely visible when installed on flip-flops. It is the intent of the appended claims to cover these and other modifications of the described embodiments as fall within the true scope and spirit of the invention.

What is claimed is:

1. A protective appliance for use with footwear of the type having a sole portion and a toe segment upstanding from the sole portion and adapted to fit between two adjacent toes of a wearer's foot, said appliance comprising:
a generally hollow body of a relatively compressible, relatively resilient material defining a hollow inner space;
said body also defining a longitudinal cut providing access to its hollow inner space;
said body being adapted to engage about the toe segment of the footwear with the toe segment positioned in the hollow inner space of said body so that said body provides a cushioned interface between the wearer's toes and the toe segment of the footwear when the footwear is worn.

2. The protective appliance of claim 1 in which the resiliency of said body is such that the longitudinal cut in said body is normally closed, is resiliently openable to allow said body to be engaged about the toe segment of the footwear, and resiliently closes after so engaged to retain said body on the toe segment.

3. The protective appliance of claim 1 in which said body comprises a relatively thin inner layer of a relatively resilient material that defines the hollow inner space of said body and a relatively thick outer layer of a relatively soft, relatively compressible material.

4. The protective appliance of claim 1 in which the hollow inner space defined by said body is sized relative to the toe segment of the footwear so that said body can move at least to a limited degree both radially and longitudinally relative to the toe segment when said body is so engaged.

5. The protective appliance of claim 2 in which said body has a unitary construction and is made of a material with sufficient compressibility to provide the cushioned interface and sufficient resiliency to retain said body on the toe segment when so engaged.

6. The protective appliance of claim 1 in which the longitudinal cut into the hollow inner space of said body is defined by overlapping segments of a side wall of said body.

7. The protective appliance of claim 6 further including releasable fastening strips for releasably retaining said side wall segments in a closed position.

8. The protective appliance of claim 1 in which said body has contoured side walls giving said body a generally hour-glass-like shape.
